(19) United States
(12) Patent Application Publication
(10) Pub. No.: US 2020/0121298 A1
(43) Pub. Date: Apr. 23, 2020

(54) ULTRASOUND DIAGNOSTIC APPARATUS

(71) Applicant: Konica Minolta, Inc., Tokyo (JP)

(72) Inventors: Yasuhiro NAKAMURA, Sagamihara-shi (JP); Tomohiko SATSUKA, Tokyo (JP)

(21) Appl. No.: 16/596,235

(22) Filed: Oct. 8, 2019

(30) Foreign Application Priority Data
Oct. 17, 2018 (JP) 2018-195497

(51) Int. Cl. A61B 8/00

(52) U.S. Cl. CPC A61B 8/546; A61B 8/56

(57) ABSTRACT

Disclosed is an ultrasound diagnostic apparatus including: a power storage that stores electric power and that releases the stored power; a first hardware processor that receives power and supplies the received power to a gel heater, in which the gel heater heats gel by the power; and a second hardware processor that receives either power supplied from an external power source or the power released from the power storage and supplies the received power to the first hardware processor. When the first hardware processor receives the power released from the power storage, the first hardware processor shuts off or reduces power supply to the gel heater.

### BACKGROUND

### Technological Field

The present invention relates to an ultrasound diagnostic apparatus.

### Description of the Related Art

In ultrasound diagnosis, examinations can be repeatedly conducted since an ultrasound examination is very safe and can provide the condition of the heart, a fetus or the like is obtained as an ultrasound image by a simple process of putting an ultrasound probe on the body surface or the inner side of the body cavity. For such ultrasound diagnosis, ultrasound diagnostic apparatuses have been known in the art. Ultrasound image data is obtained by transmitting an ultrasound from an ultrasound probe to a subject, receiving a reflected ultrasound at the ultrasound probe, and subjecting received signals to a variety of processing.

In an ultrasound examination, gel is used. In order to improve transmission of ultrasound, the gel is applied to the part of a subject, or the live body of a patient, on which an ultrasound probe is put. Since cold gel is uncomfortable for the patient, a gel warmer has been used to heat the gel to a temperature around the body temperature. Typically, the gel is stored in a plastic bottle, and the gel warmer heats the bottle as well as the gel stored therein.

In this regard, configurations for supplying electric power to a gel warmer in conventional ultrasound diagnostic apparatuses will be described referring to FIG. 7 to FIG. 9. FIG. 7 is a block diagram illustrating the functional configuration of a conventional ultrasound diagnostic apparatus 1D. FIG. 8 is a block diagram illustrating the functional configuration of a conventional ultrasound diagnostic apparatus 1E. FIG. 9 is a block diagram illustrating the functional configuration of a conventional ultrasound diagnostic apparatus 1F.

Referring to FIG. 7, the ultrasound diagnostic apparatus 1D will be described as a typical example of a conventional ultrasound diagnostic apparatus. The ultrasound diagnostic apparatus 1D includes a main body 100D, an ultrasound probe 40, and a gel warmer 70C. The main body 100D, which is a main part of the ultrasound diagnostic apparatus 1D, includes an ultrasound imaging system 10, a power controller 13D, a power storage 14A, a power button 15, and an input connector 16.

The ultrasound imaging system 10, which is a component for generating ultrasound image data and displaying an ultrasound image, includes a system controller 11, a power supply unit 12, an ultrasound signal processor 30, a display 50, and an operation interface 60. The ultrasound signal processor 30 includes a transmitter/receiver 31, a beam former 32, a signal processor 33, and a display processor 34. The ultrasound probe 40 is connected to the transmitter/receiver 31 via a cable 41.

In FIG. 7, power lines are illustrated by arrowed double lines, and signal lines for signals such as control signals are illustrated by arrowed single lines. Among the power lines, the portions to which power is being supplied are illustrated by bold lines, and the portions to which power is not being supplied are illustrated by thin lines. Further, among the components of the ultrasound diagnostic apparatus 1D, except for the ultrasound imaging system 10 and the power button 15, the components to which power is being supplied are illustrated by bold line frames, and the components to which power is not being supplied are illustrated by thin line frames. This applies to the other figures, too.

A plug of a power cable of the input connector 16 is connected to a power outlet of an external power source (commercial alternating-current (AC) power supply). Electric power of the external power source is supplied to the power controller 13D via the input connector 16. The power controller 13D receives the AC power from the input connector 16, converts the AC power to direct-current (DC) power and supplies the DC power to the power storage 14A to charge it as well as to the power supply unit 12. The power supply unit 12 supplies the power to each component of the ultrasound signal processor 30.

The gel warmer 70C heats gel by using the input AC power. The gel warmer 70C includes a power cable and a plug to be connected to an outlet of an external power source. The input connector 16 and the gel warmer 70C may be powered from the same outlet via a power strip that branches the power line. Thus, the gel warmer 70C with the plug connected to an outlet does not stop heating until the plug is disconnected from the outlet or a switch is turned off.

Referring to FIG. 8, the ultrasound diagnostic apparatus 1E will be described as another typical example of a conventional ultrasound diagnostic apparatus. The ultrasound diagnostic apparatus 1E includes a main body 100E, an ultrasound probe 40, and a gel warmer 70C. The main body 100E, which is a main part of the ultrasound diagnostic apparatus 1E, includes an ultrasound imaging system 10, a power controller 13E, a power storage 14A, a power button 15, an input connector 16, an output connector 17C, a power distributor 19E, and a switch 21.

The power distributor 19E receives power through the input connector 16 and distributes the power to the power controller 13E and the switch 21. The power controller 13E receives the AC power from the power distributor 19E, converts the AC power to DC power and supplies the DC power to the power storage 14A to charge it as well as to the power supply unit 12. Further, the power controller 13E controls ON/OFF of the switch 21 according to a control signal from the system controller 11 or the power button 15 indicating that power is ON/OFF. The output connector 17C is electrically connected to the switch 21. For example, the output connector 17C includes an outlet (socket) to which a plug of the gel warmer 70C is connectable.

When the switch 21 is ON, the switch 21 supplies AC power from the power distributor 19E to the gel warmer 70C via the output connector 17C. When the switch 21 is OFF, the switch 21 does not supply the AC power to the gel warmer 70C via the output connector 17C. That is, in response to the ultrasound diagnostic apparatus 1E being turned on, the switch 21 is turned on accordingly to supply power to the gel warmer 70C. In response to the ultrasound diagnostic apparatus 1E being turned off, the switch 21 is turned off accordingly to stop supplying power to the gel warmer 70C.

Since the ultrasound diagnostic apparatuses 1D, 1E include the respective power storages 14A, the ultrasound diagnostic apparatuses 1D, 1E may sometimes be used by being powered by the power storages 14A in a place where there is no connection to an external power source. However, when the main bodies 100D, 100E are not connected to an external power source, no power is supplied to the gel warmer 70C, and gel is not heated. To cope with the problem, an ultrasound diagnostic apparatus that supplies power to the gel warmer through a built-in USB (Universal Serial Bus) terminal has been known in the art (e.g. see JP 2011-83365A). The ultrasound diagnostic apparatus disclosed in JP 2011-83365A controls the temperature of the gel warmer to a temperature set on an operation interface according to a control program.

Referring to FIG. 9, the conventional ultrasound diagnostic apparatus 1F that supplies power to a gel warmer through a USB terminal (USB connector) will be described. The ultrasound diagnostic apparatus 1F includes a main body 100F, an ultrasound probe 40, and a gel warmer 70F. The main body 100F, which is a main part of the ultrasound diagnostic apparatus 1F, includes an ultrasound imaging system 10F, a power controller 13D, a power storage 14A, a power button 15, an input connector 16 and a USB connector 17F.

The ultrasound imaging system 10F has the same configuration as the ultrasound imaging system 10 in FIG. 7 or FIG. 8 except that the system controller 11 is replaced with a system controller 11F. In addition to the functions of the system controller 11, the system controller 11F, which is further connected to the USB connector 17F, communicates with an external device that is connected to the USB connector 17F as well as supplies power to the external device that is connected to the USB connector 17F in the form of bus power of the USB interface. The gel warmer 70F is configured to be connectable to the USB connector 17F.

When a plug of a power cable from the input connector 16 is connected to an outlet so that power is supplied from an external power source (the arrowed double bold lines in FIG. 9), the power controller 13D receives AC power from the external power source, converts the AC power to DC power and supplies the DC power to the power supply unit 12 as well as to the power storage 14A to charge it. The system controller 11F supplies power from the power supply unit 12 to the gel warmer 70F that is connected to the USB connector 17F. The system controller 11F also controls the temperature of the gel warmer 70F through the USB connector 17F.

When the plug of the power cable from the input connector 16 is not connected to an outlet so that no external power is supplied (the arrowed double thin lines in FIG. 9), the power storage 14A supplies stored power to the power controller 13D. The power controller 13D supplies the power from the power storage 14A to the power supply unit 12. The system controller 11F supplies the power from the power supply unit 12 to the gel warmer 70F that is connected to the USB connector 17F. Further, the system controller 11F controls the temperature of the gel warmer 70F through the USB connector 17F. Since it is not necessary to connect the gel warmer 70F (ultrasound diagnostic apparatus 1F) to an external power source, the burden thereof is eliminated

However, the gel warmer consumes a large amount of power. Accordingly, a problem with the conventional ultrasound diagnostic apparatus 1F is that the operable time of the ultrasound diagnostic apparatus 1F is extremely reduced in a mode where the power storage 14A supplies power to the gel warmer 70F.

### SUMMARY

It is an object of the present invention to extend the operable time in a mode where stored power is supplied to a gel warmer.

To achieve at least one of the abovementioned objects, according to a first aspect of the present invention, an ultrasound diagnostic apparatus reflecting one aspect of the present invention comprises:

a power storage that stores electric power and that releases the stored power;

a first hardware processor that receives power and supplies the received power to a gel heater, in which the gel heater heats gel by the power; and

a second hardware processor that receives either power supplied from an external power source or the power released from the power storage and supplies the received power to the first hardware processor;

wherein when the first hardware processor receives the power released from the power storage, the first hardware processor shuts off or reduces power supply to the gel heater.

According to a second aspect of the present invention, an ultrasound diagnostic apparatus reflecting one aspect of the present invention comprises:

a power storage that stores electric power and releases the stored power;

a converter that receives AC power, converts the received AC power to DC power and supplies the converted DC power to a gel heater, in which the gel heater heats gel by the DC power;

a second hardware processor that receives power supplied from an external power source or the power released from the power storage and supplies the received power to a third hardware processor, in which the third hardware processor generates ultrasound image data from a reception signal obtained by an ultrasound probe for transmitting and receiving ultrasound to and from a subject; and

a power distributor that distributes AC power supplied from the external power source to the second hardware processor and the converter.

According to a third aspect of the present invention, an ultrasound diagnostic apparatus reflecting one aspect of the present invention comprises:

a second hardware processor that supplies power to a third hardware processor, in which the third hardware processor generates ultrasound image data from a reception signal obtained at an ultrasound probe for transmitting and receiving ultrasound to and from a subject;

an uninterruptible power supply that when receiving power, supplies the received power to the second hardware processor and stores the received power, and that when receiving no power, supplies the stored power to the second hardware processor; and

a power distributor that distributes power from an external power source to the uninterruptible power supply and a gel heater, in which the gel heater heats gel by the power.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating the functional configuration of a first ultrasound diagnostic apparatus according to a first embodiment of the present invention when power is being supplied from an external power source;

FIG. 2 is a block diagram illustrating the functional configuration of the first ultrasound diagnostic apparatus when power is not being supplied from the external power source;

FIG. 3 is a block diagram illustrating the functional configuration of the first ultrasound diagnostic apparatus in a non-operating state;

FIG. 4 is a block diagram illustrating the functional configuration of a second ultrasound diagnostic apparatus according to a second embodiment when power is being supplied from an external power source;

FIG. 5 is a block diagram illustrating the functional configuration of the second ultrasound diagnostic apparatus in a non-operating state;

FIG. 6 is a block diagram illustrating the functional configuration of a third or fourth ultrasound diagnostic apparatus according to a third embodiment;

FIG. 7 is a block diagram illustrating the functional configuration of the fifth ultrasound diagnostic apparatus of the prior art;

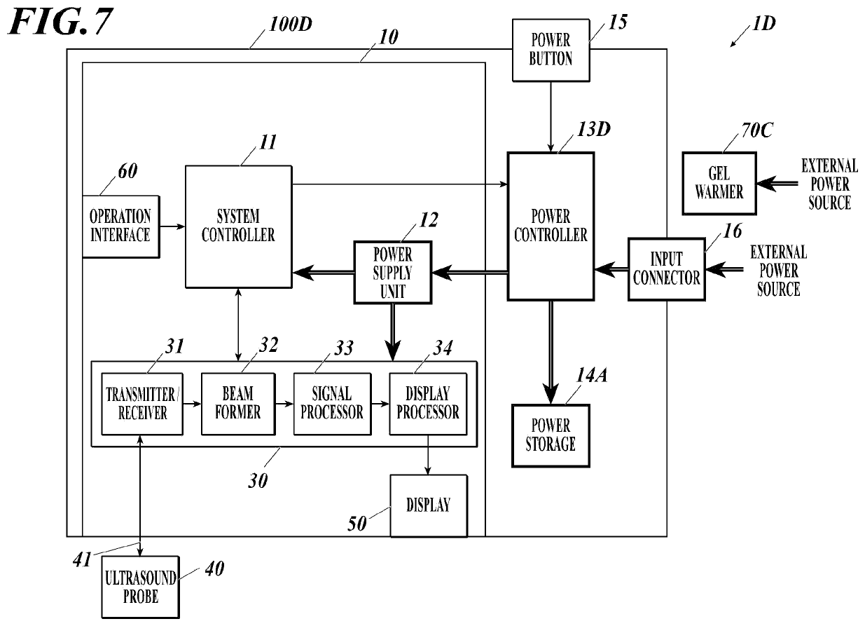

FIG. 8 is a block diagram illustrating the functional configuration of the sixth ultrasound diagnostic apparatus of the prior art; and

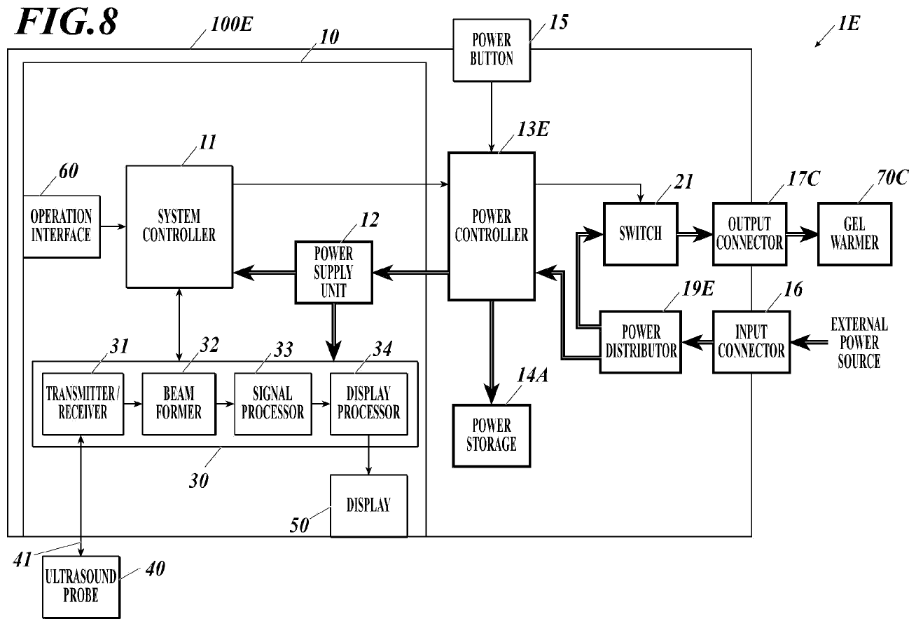

FIG. 9 is a block diagram illustrating the functional configuration of the seventh ultrasound diagnostic apparatus of the prior art.

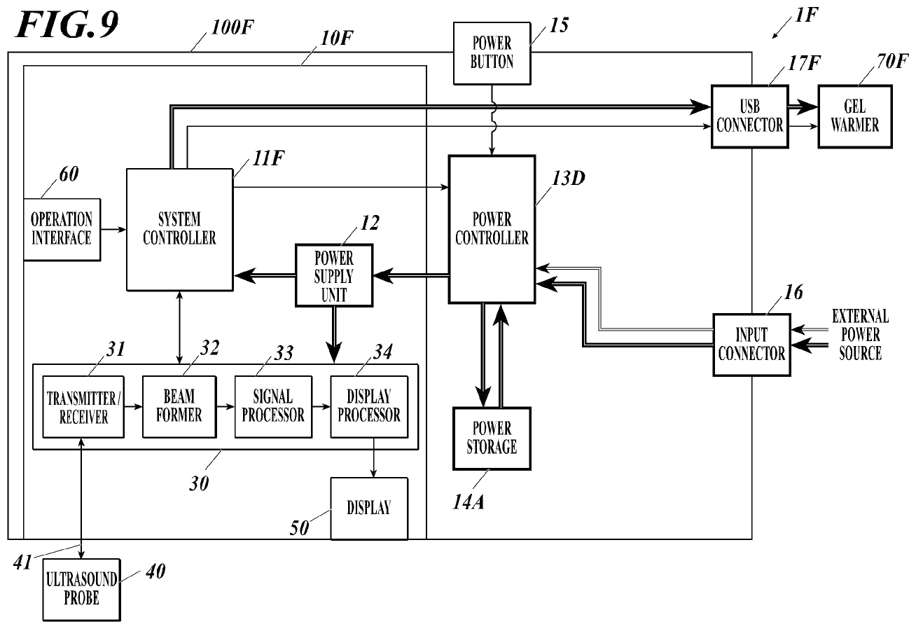

### DETAILED DESCRIPTION OF EMBODIMENTS

Hereinafter, one or more embodiments of the present invention will be described with reference to the drawings. However, the scope of the invention is not limited to the disclosed embodiments.

First to third embodiments of the present invention will be described one by one in detail referring to the drawings. However, the present invention is not limited to the illustrated examples.

### First Embodiment

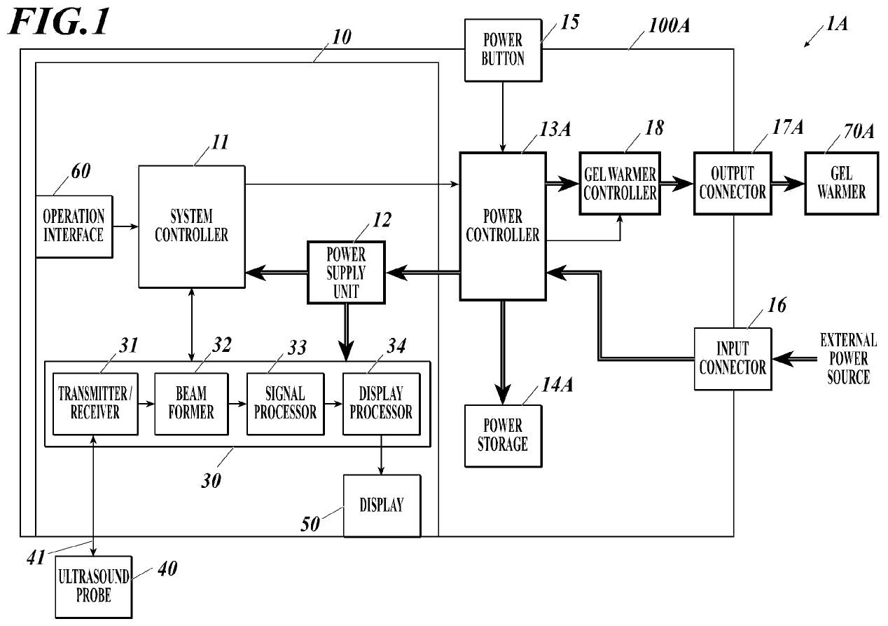

The first embodiment of the present invention will be described referring to FIG. 1 to FIG. 3. First, the device configuration of this embodiment will be described referring to FIG. 1. FIG. 1 is a block diagram illustrating the functional configuration of the ultrasound diagnostic apparatus 1A according to the embodiment when power is being supplied from an external power source.

The ultrasound diagnostic apparatus 1A of the embodiment is intended for use in a medical facility having an external power source (commercial power supply) such as a hospital. As illustrated in FIG. 1, the ultrasound diagnostic apparatus 1A includes a main body 100A, an ultrasound probe 40, and a gel warmer 70A as a gel heater.

The ultrasound probe 40 transmits ultrasound (transmission ultrasound) to a subject (not illustrated) such as the live body of a patient and receives ultrasound (reception ultrasound) including reflected ultrasound reflected at the subject and scattered ultrasound. The main body 100A is connected to an ultrasound probe 40 via a cable 41. The main body 100A sends an electric drive signal to the ultrasound probe 40 so as to cause the ultrasound probe 40 to transmit the transmission ultrasound toward the subject. Further, the main body 100A visualizes the inside of the subject as an ultrasound image based on an electric reception signal that is generated by the ultrasound probe 40 according to the reception ultrasound from the subject received at the ultrasound probe 40.

For example, transducers (not illustrated) of the ultrasound probe 40 are arranged in a one-dimensional array in the lateral direction (scanning direction). In the embodiment, for example, the ultrasound probe 40 includes 192 transducers. The transducers may be arranged in a two-dimensional array. The number of transducers may be suitably selected. Further, the ultrasound probe 40 may be of any of electrical scanning type and mechanical scanning type, and the scanning method may be any of linear scanning, sector scanning and convex scanning

The gel warmer 70A heats gel by input DC power, which includes a power cable and a connector for connection to an output connector 17A (described later). The gel warmer 70A has a function of adjusting the gel heating power according to the amount of power input. In one or more embodiments, the ultrasound diagnostic apparatus is configured to include the gel warmer. However, the ultrasound diagnostic apparatus may be configured not to include the gel warmer.

The main body 100A includes an ultrasound imaging system 10 (third hardware processor), a power controller 13A (second hardware processor), a power storage 14A, a power button 15, an input connector 16, an output connector 17A, and a gel warmer controller 18 as a gel heating controller (first hardware processor). The ultrasound imaging system 10 includes a system controller 11, a power supply unit 12, an ultrasound signal processor 30, a display 50 and an operation interface 60. The ultrasound signal processor 30 includes a transmitter/receiver 31, a beam former 32, a signal processor 33 and a display processor 34.

For example, the operation interface 60 includes a variety of switches, buttons, a track ball, a mouse and a keyboard for inputting a variety of commands such as an instruction to start examination of a subject, measurement conditions, subject information and the like. The operation interface 60 outputs operation information according to an operational input to the system controller 11. In particular, when the operator (doctor, technician, etc.) puts the ultrasound diagnostic apparatus 1A (ultrasound imaging system 10) into a non-operating state (shutdown, hibernation, sleep), the operation interface 60 receives an operator input for putting the device into the non-operating state. The term “shutdown” refers to a state in which the power of the ultrasound diagnostic apparatus 1A is off. The term “hibernation” refers to a dormant state upon which, i.e. before the power is turned off, data in a RAM (Random Access Memory) is evacuated to an auxiliary storage (not shown) such as an HDD (Hard Disk Drive), which enables resuming a halfway process by reading the data from the auxiliary storage when the device is activated again. The term “sleep” refers to a stand-by state with low power consumption in which power is supplied only to a RAM of the system controller 11 to retain currently working data in the RAM, which enables resuming a halfway process when the device again is activated again.

The transmitter/receiver 31 generates a drive signal according to a drive voltage (drive voltage value) from the system controller 11, which is an electric signal corresponding to the drive voltage, and supplies the generated drive signal to the ultrasound probe 40 through the cable 41 so as to cause the ultrasound probe 40 to generate transmission ultrasound. The transmitter/receiver 31 thus functions as a transmitter. As a transmitter, for example, the transmitter/receiver 31 includes a clock generator circuit, a delay circuit, and a pulse generator circuit. The clock generator circuit generates a clock signal for determining the transmission timing and transmission frequency of the drive signal. The delay circuit sets a delay time of the drive signal with respect to each individual pathway corresponding to each transducer. The delay circuit delays transmission of the drive signal by the set delay time so as to focus a transmission beam of the transmission ultrasound. The pulse generator circuit generates a pulse signal as the drive signal at a predetermined frequency. For example, the transmitter/receiver 31 thus configured generates transmission ultrasound by driving a part of the transducers (e.g. 64 adjacent transducers of the 192 transducers) arranged in the ultrasound probe 40. Then, the transmitter/receiver 31 shifts the active transducers in the lateral direction every time the transmitter/receiver 31 generates transmission ultrasound, so as to scan the subject. In this way, the ultrasound probe 40 transmits and receives ultrasound according to the drive signal from the transmitter/receiver 31.

Under the control of the system controller 11, the transmitter/receiver 31 also functions as a receiver that receives an electric reception signal from the ultrasound probe 40 through the cable 41. The transmitter/receiver 31 outputs the reception signal of each of the transducers of the ultrasound probe 40.

Under the control of the system controller 11, the beam former 32 generates constructive interference sound ray data from the reception signals of the transducers input from the transmitter/receiver 31. For example, the beam former 32 includes an amplifier, an analog-to-digital (A/D) converter circuit, and a delay and sum circuit. The amplifier amplifies the reception signals at a predetermined amplification factor with respect to each of individual pathways corresponding to the respective transducers. The A/D converter circuit converts the amplified reception signals by A/D conversion. The delay and sum circuits aligns the time phase of the A/D converted reception signals by adding a delay time with respect to each of the individual pathways corresponding to the respective transducers and sums (delays and sums) the aligned reception signals to generate the sound ray data.

Under the control of the system controller 11, the signal processor 33 performs envelope demodulation and logarithmic amplification on the sound ray data input from the beam former 32 and further adjusts the dynamic range and the gain. The signal processor 33 then generates a B-mode (brightness-mode) image data by conversion to brightness. That is, the B-mode image data represents the intensity of the reception signals by brightness. In addition to the B-mode image data for B-mode diagnosis, the signal processor 33 can generate ultrasound image data of other imaging modes such as A (Amplitude) mode, M (Motion) mode, a pulse-Doppler imaging, and color Doppler imaging.

Further, the signal processor 33 includes an image memory (not illustrated) composed of a semiconductor memory such as a DRAM (Dynamic RAM). Under the control of the system controller 11, the signal processor 33 stores the generated B-mode image data in the image memory on a frame basis and outputs the stored data as image data of each frame.

Under the control of the system controller 11, the display processor 34 converts the frame image data input from the signal processor 33 to image signals by coordinate conversion or the like and outputs the image signals to the display 50.

As the display 50, display devices that can be used include an LCD (Liquid Crystal Display), an organic EL (Electro-Luminescence) display, an inorganic EL display, and a plasma display. Under the control of the system controller 11 via the ultrasound signal processor 30, the display 50 displays an ultrasound image and the like on a display screen according to an image signal input from the display processor 34.

For example, the system controller 11 includes a CPU (Central Processing Unit), a ROM (Read Only Memory), and a RAM. The system controller 11 reads a variety of processing programs such as a system program stored in the ROM, develops the read program on the RAM and controls the components of the ultrasound diagnostic apparatus 1A according to the developed program. The ROM, which is constituted by a semiconductor non-volatile memory or the like, stores the system program for the ultrasound diagnostic apparatus 1A, a variety of processing programs executable on the system program and a variety of data such as a gamma table. These programs are stored in the form of program computer-readable program codes, and the CPU sequentially executes operations according to the program codes. The RAM, which is a volatile memory, provides a working area for temporarily storing a variety of programs executed by the CPU and data relating to the programs.

The power supply unit 12 supplies power to the system controller 11, the ultrasound signal processor 30, the display 50, and the operation interface 60. In particular, when in a sleep mode, the power supply unit 12 supplies power to the RAM of the system controller 11 and the power button 15 according to an instruction of the power controller 13A. The power supply unit 12 may be constituted by a regulator (DC-DC converter, series regulator). Alternatively, the regulator may be constituted by a circuit of discreet components, or an IC or a circuit module.

The power controller 13A receives power from an external power source (input connector 16) or the power storage 14A. The power controller 13A performs: supplying the power to the power supply unit 12, the power storage 14A and the gel warmer controller 18; waiting for the power button 15 to be pressed; notifying the system controller 11 of information indicating that the power button 15 has been pressed; putting the ultrasound diagnostic apparatus 1A (ultrasound imaging system 10) to a non-operating state according to the system controller 11; executing an activation process; and controlling the gel warmer controller 18. Further, the power controller 13A has a function of converting the AC power from the external power source into DC power. The power controller 13A may have any configuration depending on the complexity of control, which can be constituted by a digital circuit, a small CPU such as a microcomputer, an IC (Integrated Circuit) exclusively for power control or the like.

For example, the power storage 14A, which includes a secondary cell (battery) such as a lithium-ion cell embedded in the main body 100A, is capable of storing the DC power from the power controller 13A (charge) and releasing the DC power to the power controller 13A (discharge). Alternatively, the power storage 14A may be of different type such as a capacitor.

The power button 15, which is disposed on the surface of the main body 100A, receives an operator input of pressing down the button as an operation to turn on/off the power and outputs a corresponding operation signal to the power controller 13A. Operations that the power button 15 receives include an activating operation after the ultrasound diagnostic apparatus 1A is put into a non-operating state and a shutdown operation when the ultrasound diagnostic apparatus 1A is active (in operation).

The input connector 16, which is disposed on the surface of the main body 100A and electrically connected to the power controller 13A, is provided to be electrically connected to a power cable of a plug that is connected to an external power source (outlet of a commercial power supply). The power of the external power source is supplied to the power controller 13A through the input connector 16. The output connector 17A, which is disposed on the surface of the main body 100A and electrically connected to the gel warmer controller 18, is provided to be connected to a connector of the gel warmer 70A.

The gel warmer controller 18, which is electrically connected to the output connector 17A, allows, stops or reduces supply of the power from the power controller 13A to the gel warmer 70A through the output connector 17A under the control of the power controller 13A. The gel warmer controller 18 includes a built-in timer and can measure a predetermined period of time by using the timer. With this, the gel warmer controller 18 can supply power to the gel warmer 70A for the predetermined period of time. Specifically, the timer may be constituted by a digital circuit such as a microcomputer or by an analog circuit that measures the release time of charges accumulated in a capacitor or the like.

Regarding the components of the ultrasound diagnostic apparatus 1A, the functions of part or all of the functional blocks thereof can be achieved by a hardware circuit such as an integrated circuit. For example, the integrated circuit is an LSI (Large Scale Integration). Depending on the degree of integration, an LSI may also be referred to as an IC (Integrated Circuit), a system LSI, a super LSI, or an ultra LSI. The integrated circuit may be achieved not by an LSI but by a dedicated circuit or a general-purpose processor. An FPGA (Field Programmable Gate Array) or a reconfigurable processor that is reconfigurable in terms of connections and settings of circuit cells in an LSI may also be used. Alternatively, the functions of part or all of the functional blocks can be achieved by a software. In this case, the software is stored in at least one of a storage media such as a ROM, an optical disk, or a hard disk, and the software is executed by an arithmetic processor.

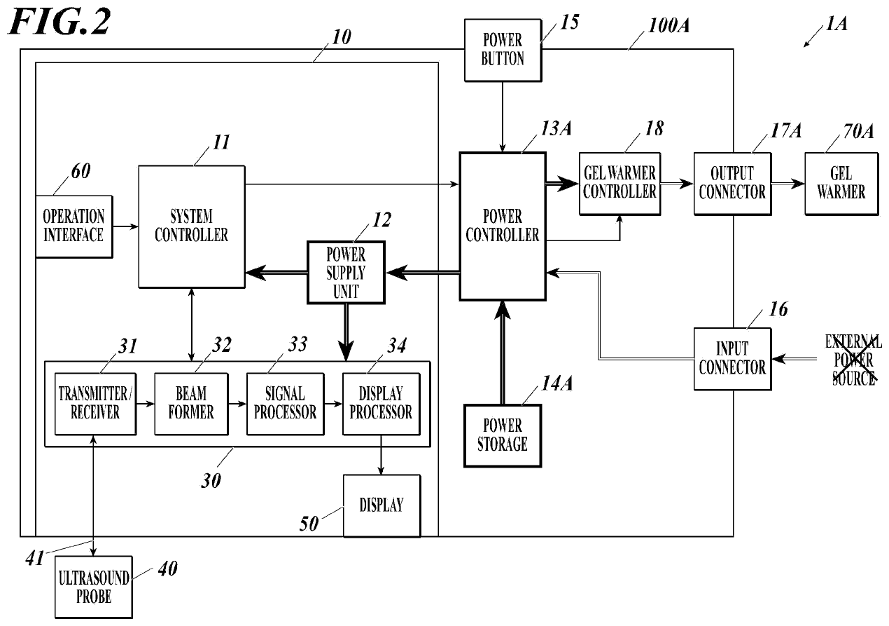

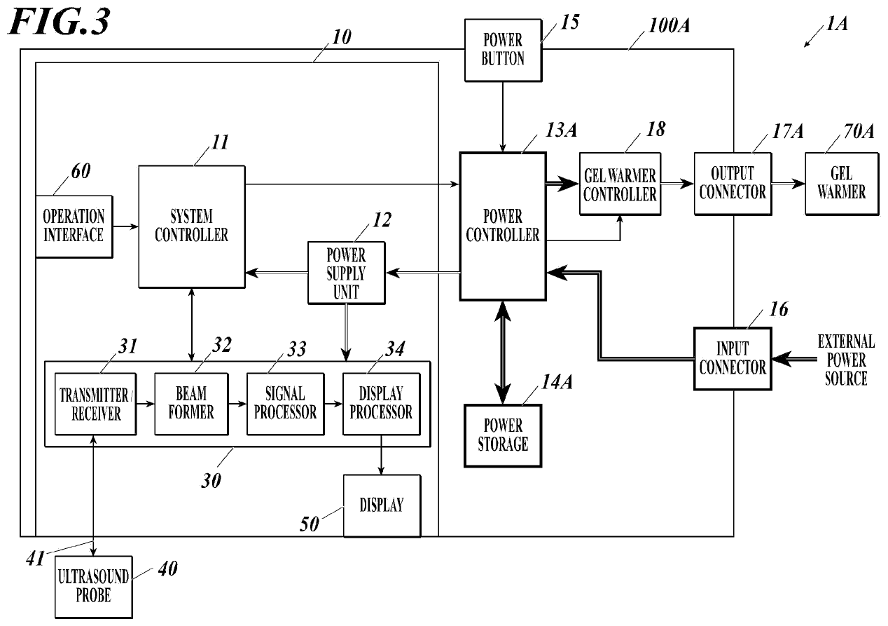

Next, the operation of the ultrasound diagnostic apparatus 1A will be described referring to FIG. 1 to FIG. 3. FIG. 2 is a block diagram illustrating the functional configuration of the ultrasound diagnostic apparatus 1A when power is not being supplied from the external power source. FIG. 3 is a block diagram illustrating the functional configuration of the ultrasound diagnostic apparatus 1A in a non-operating state.

First, the operation of the ultrasound diagnostic apparatus 1A when power is being supplied from the external power source will be described referring to FIG. 1. In the illustrated example, the ultrasound diagnostic apparatus 1A is in an operating state (a state that is not any non-operating state, i.e. a state when the power is on).

AC power of the external power source is supplied to the power controller 13A through the input connector 16. The power controller 13A detects supply of the power from the external power source. In response to the detection, the power controller 13A switches the power supply to the external power source. At the same time, the power controller 13A generates a control signal indicating that power is being supplied from the external power source, and outputs the generated signal to the gel warmer controller 18. Further, the power controller 13A converts the input AC power of the external power source to DC power and supplies the converted DC power to the power supply unit 12, the power storage 14A and the gel warmer controller 18.

The power supply unit 12 supplies the DC power from the power controller 13A to the components of the ultrasound imaging system 10. The power storage 14A is charged by the DC power supplied from the power controller 13A. According to the control signal from the power controller 13A, the gel warmer controller 18 supplies the DC power from the power controller 13A to the gel warmer 70A through the gel warmer controller 18 itself and the output connector 17A.

Next, the operation of the ultrasound diagnostic apparatus 1A when no power is being supplied from the external power source as illustrated in FIG. 2 will be described. For example, such situation occurs when the power plug of the input connector 16 of the ultrasound diagnostic apparatus 1A is temporarily disconnected from an outlet for moving the ultrasound diagnostic apparatus 1A. When power supply from the external power source is lost, the power controller 13A of the ultrasound diagnostic apparatus 1A detects absence of power supply from the external power source. In response to the detection, the power controller 13A switches the power supply to the power storage 14A. At the same time, the power controller 13A generates a control signal indicating that power is being supplied from the power storage 14A, and outputs the generated signal to the gel warmer controller 18. Further, the power controller 13A supplies the DC power (released) from the power storage 14A to the power supply unit 12 and the gel warmer controller 18.

According to the control signal from the power controller 13A, the gel warmer controller 18 supplies the DC power from the power storage 14A to the gel warmer 70A through the gel warmer controller 18 itself and the output connector 17A for a predetermined period of time by measuring the predetermined period of time with a timer. After the elapse of the predetermined period of time, the power controller 13A controls the gel warmer controller 18 to shut off the DC power at the gel warmer controller 18 (to stop the power supply to the gel warmer 70A) or to reduce the amount (voltage or current) of DC power to be supplied to the gel warmer 70A.

In this way, even after the external power source is temporarily lost, the gel warmer 70A continues heating for the predetermined period of time so that the gel does not get cold. After the elapse of the predetermined period of time, the heating by the gel warmer 70A is stopped or reduced in order to prevent or reduce discharge of the power storage 14A.

Next, the operation of the ultrasound diagnostic apparatus 1A in a non-operating state will be described referring to FIG. 3. As illustrated in FIG. 3, in the situation where power is supplied from the external power source while the ultrasound diagnostic apparatus 1A is in operation, the power controller 13A converts the AC power from the external power source to DC power and supplies the DC power to the power supply unit 12, the power storage 14A and the gel warmer controller 18 so as to charge the power storage 14A. Then, when an operator inputs an instruction to put the ultrasound diagnostic apparatus 1A (ultrasound imaging system 10) into an non-operating state (shutdown, hibernation or sleep) on the operation interface 60 or the power button 15, the system controller 11 outputs information on the designated non-operating state to the power controller 13A and puts the ultrasound imaging system 10 into the non-operating state.

According to the information on the non-operating state, the power controller 13A generates a control signal indicating that power is being supplied from an external power source and that the ultrasound imaging system 10 is in the non-operating state, and outputs the generated signal to the gel warmer controller 18. Further, the power controller 13A converts the AC power from the external power source to DC power and supplies the DC power to the power storage 14A (and to the power supply unit 12 when in a sleep mode) to charge the power storage 14A. In the sleep mode, the power supply unit 12 supplies DC power to the RAM of the system controller 11. In the case where the input control signal indicates shutdown, the gel warmer controller 18 stops supplying DC power to the gel warmer 70A, for example, because the ultrasound imaging system 10 will not perhaps be activated for a while. In the case where the input control signal indicates hibernation, the gel warmer controller 18 reduces supply of DC power to the gel warmer 70A (by reducing the voltage or the current), for example, because the ultrasound diagnostic apparatus 1A and the gel will probably be used. In the case where the input control signal indicates sleep, the gel warmer controller 18 continues supplying DC power to the gel warmer 70A without shutting off or reducing the DC power, for example, because the ultrasound diagnostic apparatus 1A and the gel will be used soon.

Then, when power supply from an external power source is lost while the ultrasound diagnostic apparatus 1A is in the non-operating state, the power controller 13A supplies the DC power from the power storage 14A to the gel warmer controller 18 according to the information on the designated non-operating state. In the sleep mode, the power controller 13A supplies the DC power to the power supply unit 12. Then, in the sleep mode, the power supply unit 12 supplies the DC power to the RAM of the system controller 11.

Further, according to the information on the designated non-operating state, the power controller 13A generates a control signal indicating that power is being supplied from the power storage 14A and that the ultrasound imaging system 10 is in the non-operating state, and outputs the generated signal to the gel warmer controller 18. Then, as in the situation where power is being supplied from an external power source, the gel warmer controller 18 allows or shuts off or reduces supply of the DC power to the gel warmer 70A according to the level or type of the non-operating state (shutdown, hibernation or sleep) specified by the control signal.

According to the embodiment, the ultrasound diagnostic apparatus 1A includes: the power storage 14A that stores power and releases the stored power; the gel warmer controller 18 that supplies input power to the gel warmer 70A that electrically heats the gel; and the power controller 13A that receives power from an external power source or the power storage 14A and outputs the received power to the gel warmer controller 18. When receiving the power that is released from the power storage 14A, the gel warmer controller 18 shuts off or reduces power supply to the gel warmer 70A.

That is, the power consumption of the gel warmer 70A is eliminated or reduced when the power stored in the power storage 14A is supplied to the gel warmer 70A. Therefore, it is possible to extend the operable time of the ultrasound diagnostic apparatus 1A.

When receiving the power released from the power storage 14A while no power is supplied from an external power source, the power controller 13A shuts off or reduces power supply to the gel warmer controller 18. That is, in the situation where the power stored in the power storage 14A is supplied to the gel warmer 70A while no power is supplied from an external power source, the power consumption of the gel warmer 70A is eliminated or reduced. Therefore, it is possible to extend the operable time of the ultrasound diagnostic apparatus 1A.

The gel warmer 70A is configured to be able to change the heating power of the gel according to the amount of power input. When receiving the power from the power storage 14A, the gel warmer controller 18 reduces the amount of power to be supplied to the gel warmer 70A. Therefore, it is possible to save power consumption while weakly heating the gel so that the gel does not completely get cold.

The gel warmer controller 18 shuts off or reduces power supply to the gel warmer 70A after the elapse of the predetermined time since power supply from an external power source is lost. Therefore, it is possible to save power while heating the gel for the predetermined period of time so that the gel does not get cold.

The power controller 13A outputs to the gel warmer controller 18 a control signal indicating whether power is being supplied from an external power source or the power storage 14A. According to the control signal, the gel warmer controller 18 shuts off or reduces power supply to the gel warmer 70A when receiving power from the power storage 14A. Therefore, it is possible to readily switch the power supply to the gel warmer 70A between allowed and shut-off or reduced according to the power supplying state specified by the control signal.

The ultrasound diagnostic apparatus 1A includes the ultrasound imaging system 10 that generates ultrasound image data from signals received from the ultrasound probe 40 that transmits and receives ultrasound to and from a subject. When the ultrasound imaging system 10 is in a non-operating state, the gel warmer controller 18 allows or shuts off or reduces power supply to the gel warmer 70A according to the level or type of the non-operating state. Therefore, when the ultrasound imaging system 10 is in a non-operating state, it is possible to reduce power supply to the gel warmer 70A in harmony with the non-operating state. By stopping or reducing power supply to the gel warmer 70A when in a non-operating state, it is possible to reduce evaporation of the gel as well as discharge of the power storage 14A.

The non-operating state is shutdown, hibernation or sleep. When in a non-operating state that is shutdown, it is possible to reduce evaporation of the gel and discharge of the power storage 14A, for example, by stopping power supply to the gel warmer 70A. When in a non-operating state that is hibernation, it is possible to weakly heat the gel, for example, by reducing power supply to the gel warmer 70A. When in a non-operating state that is sleep, it is possible to keep the gel ready to use as well as the ultrasound imaging system 10, for example, by continuing power supply to the gel warmer 70A.

The power storage 14A includes a battery. Therefore, it is possible to suitably store and release electric power.

### Second Embodiment

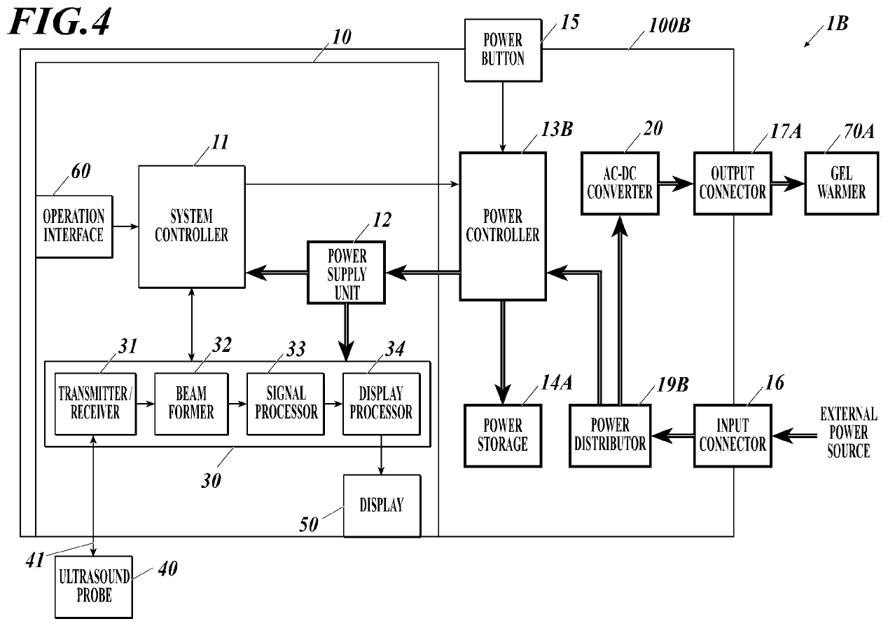

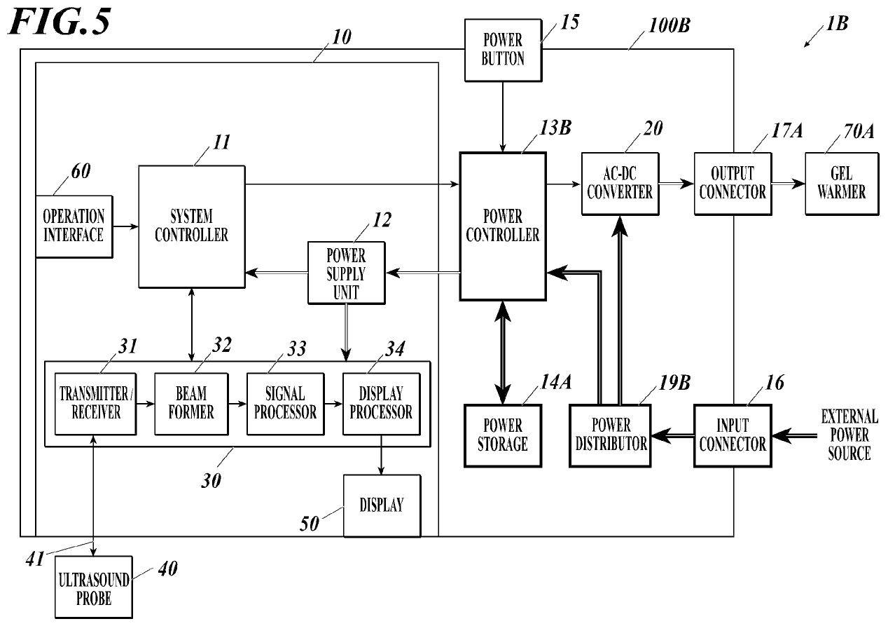

A second embodiment of the present invention will be described referring to FIG. 4 and FIG. 5. FIG. 4 is a block diagram illustrating the functional configuration of an ultrasound diagnostic apparatus 1B according to this embodiment when power is being supplied from an external power source. FIG. 5 is a block diagram illustrating the functional configuration of the ultrasound diagnostic apparatus 1B in a non-operating state.

In this embodiment, the ultrasound diagnostic apparatus 1B has the configuration as illustrated in FIG. 4. In the ultrasound diagnostic apparatus 1B, the same components as those in the ultrasound diagnostic apparatus 1A of the first embodiment are denoted by the same reference signs, and the description thereof is omitted.

As illustrated in FIG. 4, the ultrasound diagnostic apparatus 1B includes a main body 100B, an ultrasound probe 40, and a gel warmer 70A. The main body 100B includes an ultrasound imaging system 10 (third hardware processor), a power controller 13B (second hardware processor), a power storage 14A, a power button 15, an input connector 16, an output connector 17A, a power distributor 19B, an AC-DC converter 20 as a power converter.

The power controller 13B receives power from an external power source (input connector 16) or the power storage 14A. The power controller 13B performs: supplying the power to the power supply unit 12, and the power storage 14A; waiting for the power button 15 to be pressed; notifying the system controller 11 of information indicating that the power button 15 has been pressed; putting the ultrasound diagnostic apparatus 1B into a non-operating state according to the system controller 11; executing an activation process; and controlling the AC-DC converter 20. Further, the power controller 13B has a function of converting AC power from the external power source to DC power.

The power distributor 19B receives power from the external power source through the input connector 16 and distributes the power to the power controller 13B and AC-DC converter 20. The AC-DC converter 20 is a circuit that receives the AC power from the external power source through the power distributor 19B, converts the AC power to DC power and outputs the DC power to the output connector 17A.

Next, the operation of the ultrasound diagnostic apparatus 1B will be described referring to FIG. 4 and FIG. 5. First, the operation of the ultrasound diagnostic apparatus 1B when power is being supplied from the external power source will be described referring to FIG. 4.

In the example illustrated in FIG. 4, the ultrasound diagnostic apparatus 1B is in operation. The AC power from the external power source is supplied to the power controller 13B and the AC-DC converter 20 through the input connector 16 and the power distributor 19B. The power controller 13B detects power supply from the external power source. If there is power supply from the external power supply, the power controller 13B switches the power supply to the external power source. At the same time, the power controller 13B converts the DC power from the external power source to AC power and supplies the DC power to the power supply unit 12 and the power storage 14A.

The AC-DC converter 20 converts the AC power from the external power source to DC power and supplies the DC power to the gel warmer 70A through the output connector 17A.

As with the ultrasound diagnostic apparatus 1A according to the first embodiment, when the power supply from the external power source is lost, the power controller 13B of the ultrasound diagnostic apparatus 1B supplies DC power from the power storage 14B to the power supply unit 12. Further, since the power supply from an external power source is lost, no power is supplied to the AC-DC converter 20, and no power is supplied to the gel warmer 70A accordingly. Therefore, the gel is not heated.

Next, the operation of the ultrasound diagnostic apparatus 1B in a non-operating state will be described referring to FIG. 5. As illustrated in FIG. 5, in the situation where power is supplied from the external power source to the ultrasound diagnostic apparatus 1B, the AC power of the external power source is supplied to the power controller 13B through the input connector 16 and the power distributor 19B. The power controller 13B converts the AC power from the external power source to DC power and supplies the DC power to the power supply unit 12 and the power storage 14A to charge the power storage 14A. Then, in response to the operator inputting an instruction to put the ultrasound diagnostic apparatus 1B (ultrasound imaging system 10) into a non-operating state on the operation interface 60 or the power button 15, the system controller 11 outputs information on the designated non-operating state to the power controller 13B and puts the ultrasound imaging system 10 into the non-operating state.

In response to receiving the information on the non-operating state, the power controller 13B detects power supply from the external power source. If there is power supply from the external power supply, the power controller 13B generates a control signal indicating that power is being supplied from the external power source and that the apparatus is in the non-operating state, and outputs the control signal to the AC-DC converter 20. According to the control signal, the AC-DC converter 20 shuts off supply of the DC power, which is converted from the AC power from the external power source through the input connector 16 and the power distributor 19B, to the gel warmer 70A through the output connector 17A. As a result, the gel is not heated by the gel warmer 70A.

When power supply from the external power source is lost while the ultrasound diagnostic apparatus 1B is in a non-operating state, the power controller 13B supplies DC power from the power storage 14A to the power supply unit 12. Further, since the power supply from the external power source is lost, no power is supplied to the AC-DC converter 20, and no power is supplied to the gel warmer 70A accordingly. As a result, the gel is not heated by the gel warmer 70A.

According to the embodiment, the ultrasound diagnostic apparatus 1B includes: the power storage 14A that stores power and releases the stored power; the AC-DC converter 20 that outputs converted DC power to the gel warmer 70A that heats the gel by using the DC power; the power controller 13B that outputs power from an external power source or power from the power storage 14A to the ultrasound imaging system 10; and the power distributor 19B that distributes the AC power from the external power source to the power controller 13B and the AC-DC converter 20.

In this configuration, when power is being supplied from the power storage 14A to the power controller 13B, the power consumption of the gel warmer 70A is eliminated Therefore, it is possible to extend the operable time of the ultrasound diagnostic apparatus 1B.

When the ultrasound imaging system 10 is in a non-operating state, the power controller 13B causes the AC-DC converter 20 to shut off power supply to the gel warmer 70A. When the ultrasound imaging system 10 is in a non-operating state, it is possible to control power supply to the gel warmer 70A in harmony with the non-operating state. By stopping the power supply to the gel warmer 70A when in a non-operating state, it is possible to reduce evaporation of the gel as well as power consumption.

### Third Embodiment

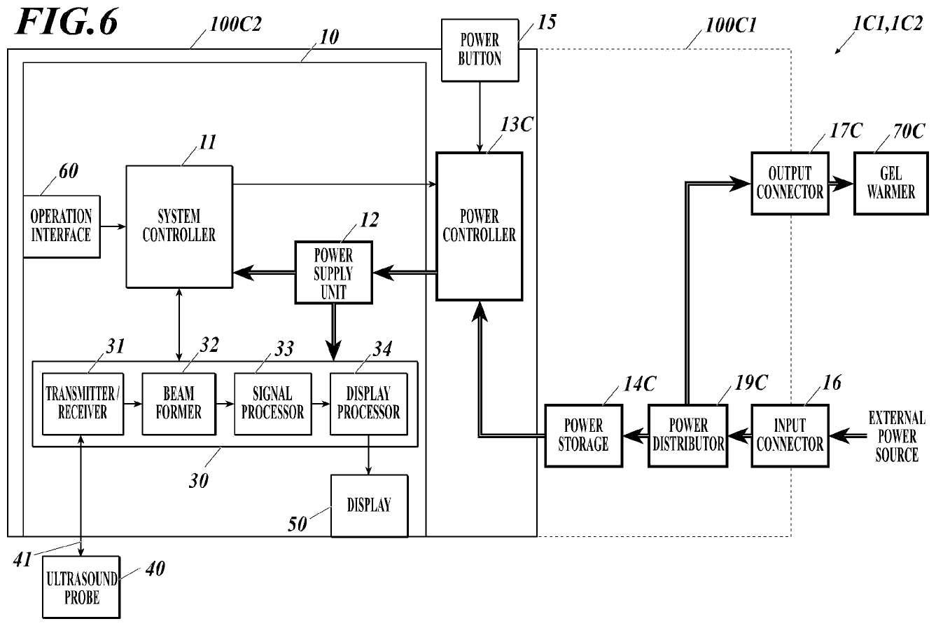

A third embodiment of the present invention will be described referring to FIG. 6. FIG. 6 is a block diagram illustrating the functional configuration of an ultrasound diagnostic apparatus 1C1 or 1C2 according to the embodiment.

In this embodiment, the ultrasound diagnostic apparatus 1C1 or 1C2 has the configuration as illustrated in FIG. 6. In the ultrasound diagnostic apparatus 1C1 or 1C2, the same components as those in the ultrasound diagnostic apparatus 1A of the first embodiment are denoted by the same reference signs, and the description thereof is omitted.

As illustrated by the solid lines and the dotted lines in FIG. 6, the ultrasound diagnostic apparatus 1C1 includes a main body 100C1, an ultrasound probe 40, and a gel warmer 70C. The main body 100C1 includes an ultrasound imaging system 10 (third hardware processor), a power controller 13C (second hardware processor), a power storage 14C, a power button 15, an input connector 16, an output connector 17C, and a power distributor 19C.

The power distributor 19C receives power from an external power source through the input connector 16 and distributes the power to the power storage 14C and the output connector 17A. The output connector 17C is disposed on the surface of the main body 100C1. The output connector 17C is electrically connected to the power distributor 19C and includes an outlet to which a power plug of the gel warmer 70C is connected.

The power storage 14C is constituted by an uninterruptible power supply (UPS), which is equipped with a power storage unit for storing power such as a secondary battery (cell). The power storage 14C is capable of supplying power at a predetermined output level for a certain period of time after power supply from the external power source is lost. That is, as well as receiving power from the external power source through the power distributor 19C and supplying the power to the power controller 13C, the power storage 14C also supplies DC power from the power storage unit to the power controller 13C when the power plug of the ultrasound diagnostic apparatus 1C1 is temporarily disconnected from an outlet of the external power source so that AC power is not supplied from the external power source through the power distributor 19C.

The power controller 13C receives power from the power storage 14C. The power controller 13 performs: supplying the power to the power supply unit 12; waiting for the power button 15 to be pressed; notifying the system controller 11 of information indicating that the power button 15 has been pressed; putting the ultrasound diagnostic apparatus 1C into a non-operating state according to the system controller 11; and executing an activation process. Further, the power controller 13 C has a function of converting AC power from the power storage 14C to DC power.

As an alternative configuration from that of the ultrasound diagnostic apparatus 1C1, the ultrasound diagnostic apparatus 1C2 as illustrated by the solid line in FIG. 6 may be provided instead. The ultrasound diagnostic apparatus 1C2 includes a main body 100C2, an ultrasound probe 40, a power storage 14C, an input connector 16, an output connector 17C, a power distributor 19C, and a gel warmer 70C. The main body 100C2 includes an ultrasound imaging system 10, a power controller 13C, and a power button 15. In the ultrasound diagnostic apparatus 1C2, the power storage 14, the input connector 16, the output connector 17C, the power distributor 19C and the gel warmer 70C are configured as separate components from the main body 100C2. For example, these components are disposed on a seat (cart) for mounting the main body 100C2 and the like.

Next, the operation of the ultrasound diagnostic apparatus 1C1 will be described referring to FIG. 6. First, the operation of the ultrasound diagnostic apparatus 1C1 when power is being supplied from an external power source will be described.

In the example illustrated in FIG. 6, the ultrasound diagnostic apparatus 1C1 is in operation. AC power of the external power source is supplied to the power storage 14C and the output connector 17A through the input connector 16 and the power distributor 19C. The gel warmer 70A heats gel by being supplied with AC power from the external power source through the output connector 17A.

The power storage 14C receives AC power of the external power source from the power distributor 19C and supplies the AC power to the power controller 13C. The power controller 13C converts the supplied AC power of the external power source to DC power and supplies the DC power to the power supply unit 12.

When the power supply from the external power source is lost in the ultrasound diagnostic apparatus 1C1, the power storage 14C supplies DC power from the power storage unit to the power controller 13C. The power controller 13C supplies the DC power from the power storage 14C to the power supply unit 12. Since no power is supplied to the gel warmer 70C, the gel is not heated.

The operation of the ultrasound diagnostic apparatus 1C2 is the same as that of the above-described ultrasound diagnostic apparatus 1C1.

According to the embodiment, the ultrasound diagnostic apparatus 1C1 or 1C2 includes: the power controller 13C that supplies power to the ultrasound imaging system 10; the power storage 14C that, when receiving power, outputs the received power to the power controller 13C and stores the power and that, when receiving no power, outputs the stored power to the power controller 13C; and the power distributor 19C that distributes power from the external power source to the power storage 14C and the gel warmer 70C for electrically heating the gel.

In this configuration, when the power stored in the power storage 14C is supplied to the power controller 13C, the power consumption of the gel warmer 70C can be eliminated Therefore, it is possible to extend the operable time of the ultrasound diagnostic apparatus 1C1 or 1C2.

The ultrasound diagnostic apparatus 1C2 includes the main body 100C2 that includes the power controller 13C. Further, the power storage 14C and the power distributor 19C are disposed outside the main body 100C2. Therefore, it is possible reduce the size of the main body 100C2.

The above description for the embodiments merely illustrate suitable examples of ultrasound diagnostic apparatuses according to the present invention, and the present invention is not limited thereto. For example, at least two of the configurations of the above-described embodiments may be combined.

Further, suitable changes can be made in detailed configurations and detailed operations of the components of the above-described ultrasound diagnostic apparatuses 1A, 1B, 1C1, 1C2 according to the embodiments without departing from the features of the present invention.

Although embodiments of the present invention have been described and illustrated in detail, the disclosed embodiments are made for purposes of illustration and example only and not limitation. The scope of the present invention should be interpreted by terms of the appended claims

The entire disclosure of Japanese patent Application No.2018-195497, filed on 17 Oct. 2018, is incorporated herein by reference in its entirety.

What is claimed is:

1. An ultrasound diagnostic apparatus, comprising:
a power storage that stores electric power and that releases the stored power;
a first hardware processor that receives power and supplies the received power to a gel heater, in which the gel heater heats gel by the power; and
a second hardware processor that receives either power supplied from an external power source or the power released from the power storage and supplies the received power to the first hardware processor;
wherein when the first hardware processor receives the power released from the power storage, the first hardware processor shuts off or reduces power supply to the gel heater.

2. The ultrasound diagnostic apparatus according to claim 1, wherein when no power is supplied from the external power source and the second hardware processor receives the power released from the power storage, the second hardware processor shuts off or reduces the power supply to the gel heater.

3. The ultrasound diagnostic apparatus according to claim 1,
wherein the gel heater is capable of changing a heating power of the gel according to an amount of power received, and
wherein when the first hardware processor receives the power released from the power storage, the first hardware processor reduces an amount of power supplying to the gel heater.

4. The ultrasound diagnostic apparatus according to claim 1, wherein the first hardware processor shuts off or reduces the power supply to the gel heater after elapse of a predetermined period of time since power supply from the external power source is lost.

5. The ultrasound diagnostic apparatus according to claim 1,
wherein the second hardware processor outputs a control signal to the first hardware processor, the control signal indicating a power supply status of whether power is supplied from the external power source or from the power storage, and
wherein when the first hardware processor receives the power released from the power storage, the first hardware processor shuts off or reduces power supply to the gel heater according to the power supply status indicated by the control signal.

6. The ultrasound diagnostic apparatus according to claim 1, further comprising:
a third hardware processor that generates ultrasound image data from a reception signal obtained by an ultrasound probe for transmitting and receiving ultrasound to and from a subject,
wherein when the third hardware processor is in an non-operating state, the first hardware processor allows, shuts off or reduces the power supply to the gel heater according to the non-operating state.

7. An ultrasound diagnostic apparatus, comprising:
a power storage that stores electric power and releases the stored power;
a converter that receives AC power, converts the received AC power to DC power and supplies the converted DC power to a gel heater, in which the gel heater heats gel by the DC power;
a second hardware processor that receives power supplied from an external power source or the power released from the power storage and supplies the received power to a third hardware processor, in which the third hardware processor generates ultrasound image data from a reception signal obtained by an ultrasound probe for transmitting and receiving ultrasound to and from a subject; and
a power distributor that distributes AC power supplied from the external power source to the second hardware processor and the converter.

8. The ultrasound diagnostic apparatus according to claim 7, wherein when the third hardware processor is in an non-operating state, the second hardware processor causes the converter to shut off power supply to the gel heater.

9. The ultrasound diagnostic apparatus according to claim 6, wherein the non-operating state is any one of shutdown, hibernation and sleep.

10. The ultrasound diagnostic apparatus according to claim 8, wherein the non-operating state is any one of shutdown, hibernation and sleep.

11. The ultrasound diagnostic apparatus according to claim 1, wherein the power storage comprises a battery.

12. The ultrasound diagnostic apparatus according to claim 7, wherein the power storage comprises a battery.

13. An ultrasound diagnostic apparatus, comprising:
a second hardware processor that supplies power to a third hardware processor, in which the third hardware processor generates ultrasound image data from a reception signal obtained at an ultrasound probe for transmitting and receiving ultrasound to and from a subject;
an uninterruptible power supply that when receiving power, supplies the received power to the second hardware processor and stores the received power, and that when receiving no power, supplies the stored power to the second hardware processor; and
a power distributor that distributes power from an external power source to the uninterruptible power supply and a gel heater, in which the gel heater heats gel by the power.

14. The ultrasound diagnostic apparatus according to claim 13, further comprising: a main body that comprises the second hardware processor,
wherein the uninterruptible power supply and the power distributor are disposed outside the main body.
